(19) United States
(12) Patent Application Publication
(10) Pub. No.: US 2012/0305787 A1
(43) Pub. Date: Dec. 6, 2012

(54) Apparatus and Method for Area Disinfection Using Ultraviolet Light

(76) Inventor: Brian Roy Henson, Rockwood (CA)

(21) Appl. No.: 13/153,408

(22) Filed: Jun. 4, 2011

(51) Int. Cl. B01J 19/12; G01J 1/42

(52) U.S. Cl. 250/372; 250/492.1

(57) ABSTRACT

A surface disinfection system comprising a plurality of independently placeable and controllable portable ultraviolet light emitting assemblies (ULAs), and a control station for remotely controlling the plurality of light assemblies. A cart housing the control station includes a dock for storing and transporting the assemblies. Each assembly includes a tubular UV-C lamp mounted on a portable base unit that includes electronic components for generating power to the lamp, detecting motion within the room being sterilized, detecting fluence levels and for audible alarm, and for wireless communication with the control station which is located outside the room during operation of the system. Using a plurality of ULAs permits strategic placement of the radiation sources to minimize shadows and thereby provides a thorough degree of disinfection. Independent control of the ULAs permits shutting down any unit to minimize the exposure to which UV-degradable materials are subject by repeated disinfections over time.

### TECHNICAL FIELD

The present invention relates to an apparatus and method for disinfecting surfaces; more particularly, to an apparatus and method for disinfecting surfaces using ultraviolet light; and most particularly, to an improved apparatus and method for the disinfection of rooms in, for example, hospitals and clinics, using UV-C light.

### BACKGROUND OF THE INVENTION

Surface disinfection of patient care areas is a key factor in the constant battle to reduce or eliminate Hospital Acquired Infections (HAIs), also known in the art as nosocomial diseases or infections. Increased evidence published in scientific literature confirms that Clostridium difficile, MRSA, VRE, Acinetobacter baumannii, Bacillus subtilis var. niger, Bacillus anthracis Sterne, and influenza are transmitted via environmental surfaces and air. The problem has become so serious that many hospitals must close critical areas, such as operation theaters and intensive care units, to eradicate pathogens via terminal cleaning. HAIs contribute to rising health care costs and can lead to severe, if not lethal, affects on patients.

Surface disinfection of patient care areas can be performed by exposing surfaces to a dose (also referred to herein as “fluence”) of UV-C light, which is a form of electromagnetic radiation that is harmful to micro-organisms such as pathogens, viruses, and molds. Fluence is a measure of the quantity of light or other radiation impinging from all directions on the smallest possible three dimensional object. Fluence is often expressed in millijoules per square centimeter (mJ/cm²).

Ultraviolet germicidal irradiation (UVGI) is a sterilization method that uses ultraviolet (UV) radiation at a sufficiently short wavelength to break down micro-organisms. The short wavelength of UV-C is harmful to forms of life at the micro-organic level by destroying nucleic acids in these organisms so that their DNA and/or RNA chemical structure is disrupted by the UV radiation. The disruption prevents micro-organisms from replicating, thereby rendering them inactive and unable to cause infection. The primary mechanism of inactivation by UV is the creation of pyrimidine dimers which are bonds formed between adjacent pairs of thymine or cytosine pyrimidines on the same DNA or RNA strand.

Low pressure mercury lamps are particularly suitable for disinfection applications because they emit two narrow peak wavelengths of light at 185 nm and 254 nm, the latter peak being close to the wavelength where DNA and RNA experience maximum UV absorption (253.7 nm). The 185 nm emission causes disassociation of oxygen molecules to create ozone, a gas with a short half life that is an air pollutant with harmful effects on respiratory systems. The EPA has designated a safe concentration of ozone concentration to be 0.05 ppm in air. Therefore, UV lamps that generate ozone are generally undesirable for use in closed areas.

A primary benefit of using UV light for disinfection is that it does not contain or create any residuals or byproducts, such as can occur with chemical methods of purification. In fact, UV light is sometimes used to remove residuals, and disinfection by-products, such as chlorine, peroxide, ozone, and trihalomethanes, that can result from other purification processes.

Different species of microorganisms require varying levels of UV-C exposure, but nearly all can be effectively inactivated with a fluence level of about 30 mJ/cm² of surface area. Fluence levels of this intensity can achieve a 4-log reduction for most microorganisms, equivalent to a 99.99% reduction in the number of active organisms.

However, the effectiveness of UV surface disinfection is dependent on line of sight exposure of the micro-organisms to the UV source. Environments with obstacles that block the source are not as effective, and UV reflectance may be low and unreliable. In such an environment, effectiveness is then reliant on the placement of the source system so that line-of-sight is optimum for surface disinfection. The effectiveness of a surface disinfection unit (SDU) in such an environment depends on a number of factors, including the length of time a micro-organism is exposed to UV; power fluctuations of the UV source that impact the wavelength; the distance of the surface from the radiation source; the ambient temperature; the humidity of the air; the presence of particles in the air; the presence of dust and dirt on the lamp surface; the presence of particles that can protect the micro-organisms from UV; and a microorganism's ability to withstand UV during its exposure.

A portable room disinfection unit employing a plurality of UV lamps on a single base unit is available under the trade name TRU_D from Lumalier Corporation, Memphis, Tenn., USA. The tubular lamps are disposed vertically about a vertical axis and irradiate radially in different directions. A shortcoming of such a single-unit ultraviolet area sterilizer (UVAS) is that any equipment or appurtenances in the room, such as beds, tables, and chairs must necessarily create shadow areas which can be irradiated only at a reduced intensity by reflections.

U.S. Pat. Nos. 6,656,424 and 6,911,177 disclose a method and apparatus for a mobile or stationary automated UVAS. The UVAS is positioned in a room, such as an operating room or intensive care unit, where concern exists regarding the presence of pathogenic bacteria on environmental surfaces. For an initial interval after actuation, motion detectors sense movement, to assure that personnel have evacuated the space to be sterilized. Subsequently, UV-C generators, such as a bank of mercury bulbs, generate intense levels of UV-C. After the bulbs have reached a steady state of output, an array of UV-C sensors scan the room to determine the darkest area, or the area reflecting the lowest level of UV-C back to the sensors. A basic stamp contained in the device calculates the time required to obtain a bactericidal dose of UV-C reflected back from darkest area. The UVAS transmits the calculated dose of UV-C, as well as other monitoring information, to the remote control where it is displayed to the user. Once a bactericidal dose has been reflected to all the sensors, the unit notifies the user and shuts down. By relying on reflected doses rather than direct exposure, the UVAS purportedly is able to sterilize or sanitize all surfaces within the room that are within view of an exposed wall or ceiling.

As noted above for the TR_D UVAS, a shortcoming of such a single-unit UVAS is that any equipment or appurtenances in the room, such as beds, tables, and chairs must necessarily create shadow areas which can be irradiated only at a reduced intensity by reflections. Further, auxiliary spaces, such a bathroom that commonly accompanies a hospital room, cannot be properly irradiated by a single UVAS, so a second UVAS is required. The patent references disclose an embodiment wherein a second UV lamp may be disposed apart from the UVAS, but powered and controlled by the UVAS, to assist in irradiating shadow areas of the UVAS.

What is needed in the art is a UV surface disinfection system comprising a plurality of independently placeable and independently controllable surface disinfection units controlled by a single remote control console, each unit having but a single ultraviolet lamp. The present invention fulfills this need as well as other needs.

### BRIEF SUMMARY OF THE INVENTION

Briefly described, the present invention is generally directed to an ultraviolet light emitting assembly (ULA) for disinfecting surfaces within an area, such as a room in a hospital or clinic. The ULA may comprise a base unit including a programmable logic controller and a ventilation fan, a support rail mounted to the base unit, a transparent sleeve supported by the support rail, and a UV-C light emitting source positioned within the sleeve defining a ventilation channel between the sleeve and the UV-C light emitting source. The ventilation channel may include a first opened end and a second opened end. The ULA may also include a temperature sensor associated with the UV-C light emitting source for monitoring the temperature of the UV-C light emitting source. The ventilation fan is positioned within the base unit so that it may blow air into the first opened end of the ventilation channel. The programmable logic controller is communication with the temperature sensor an is configured for enabling the ventilation fan if the temperature of the UV-C light emitting source exceeds a predetermined threshold. The ventilation fan may also be used to maintain the temperature of the UV-C light emitting source within a predetermined temperature range. Further, the programmable logic controller may be configured for selectively enabling the UV-C light emitting source to radiate UV-C light into the area to disinfect the surfaces located within the area

The present invention is also directed to a surface disinfection system comprising a plurality of independently placeable and independently controllable portable ULAs, and a control station for remotely controlling activation and deactivation of the plurality of light assemblies by wireless data communication. Each ULA includes a tubular lamp, for example, an amalgam lamp, vertically oriented and positioned within a protective quartz sleeve, wherein the lamp is capable of UV-C emission at a wavelength of about 254 nm. The lamp is mounted on a portable base unit, preferably with lockable castors for easy positioning of the ULA. Within the base unit are electronic components for generating power to the lamp, for detecting motion within the room being sterilized, for detecting fluence levels, for generating an audible alarm, and for wireless communication with a control station which is located remotely outside the room during operation of the system. Each base unit preferably may be connected directly for power to a 110v AC room receptacle. The plurality of ULAs permits strategic placement of the light emitting sources to minimize shadows and thereby provides a more thorough degree of disinfection than is possible with a single-location device as in the prior art. Further, independent control of the plurality of ULAs permits shutting down one such unit operating in an area requiring less lengthy irradiation, such as a small bathroom. This minimizes the total exposure to which UV-degradable materials may be subject by repeated disinfections over time. In one embodiment, a portable cart housing the control station includes a dock for storing and transporting the plurality of base units between uses.

In operation, the ULAs are unloaded from the cart and positioned strategically within the area to be irradiated, taking into account various factors including room width and length and the minimization of shadows when the ULAs are energized. The ULAs are plugged in, and the cart is rolled outside the area. The area is checked for absence of all personnel and then temporarily sealed against re-entry. The control station is programmed for operation of the ULAs and the ULAs are energized. At the conclusion of the programmed time period, the ULAs are denergized and the area is opened for re-entry of personnel. The ULAs are unplugged and reloaded onto the cart for future use.

### BRIEF DESCRIPTION OF THE DRAWINGS

The accompanying drawings form a part of this specification and are to be read in conjunction therewith, wherein like reference numerals are employed to indicate like parts in the various views, and wherein:

FIG. 1 is an elevational view of one embodiment of an ultraviolet light emitting assembly (ULA) in accordance with the present invention;

FIG. 2 is a perspective view of the ULA shown in FIG. 1;

FIG. 3 is an enlarged view of a base unit of the ULA shown in FIG. 2 with the protective sleeve removed;

FIG. 4 is a view of the base unit similar to FIG. 3 with a portion of the outer case removed showing various components of the ULA;

FIG. 5 is a cross-sectional view of the lower portion of the ULA taken along line 5-5 in FIG. 2;

FIG. 6 is a cross-sectional view of the upper portion of the ULA taken along line 6-6 in FIG. 2;

FIG. 7 is a perspective view of another embodiment of a ULA in accordance with the present invention, showing three ULAs docked on a cart for storage and transport, wherein the cart also includes the control station for the ULAs;

FIG. 8 is a perspective view of one of the ULAs shown in FIG. 7;

FIG. 9 is an enlarged view of the base unit of the ULA shown in FIG. 8;

FIG. 10 is an enlarge view of a foot pedal on the docking cart shown in FIG. 7;

FIG. 11 is a perspective view of the control station on the docking cart shown in FIG. 7;

FIG. 12 is a schematic view of another embodiment of the cart shown in FIG. 7.

FIG. 13 is a schematic plan view of a system including an example of a multiple-ULA layout for use in accordance with the present invention;

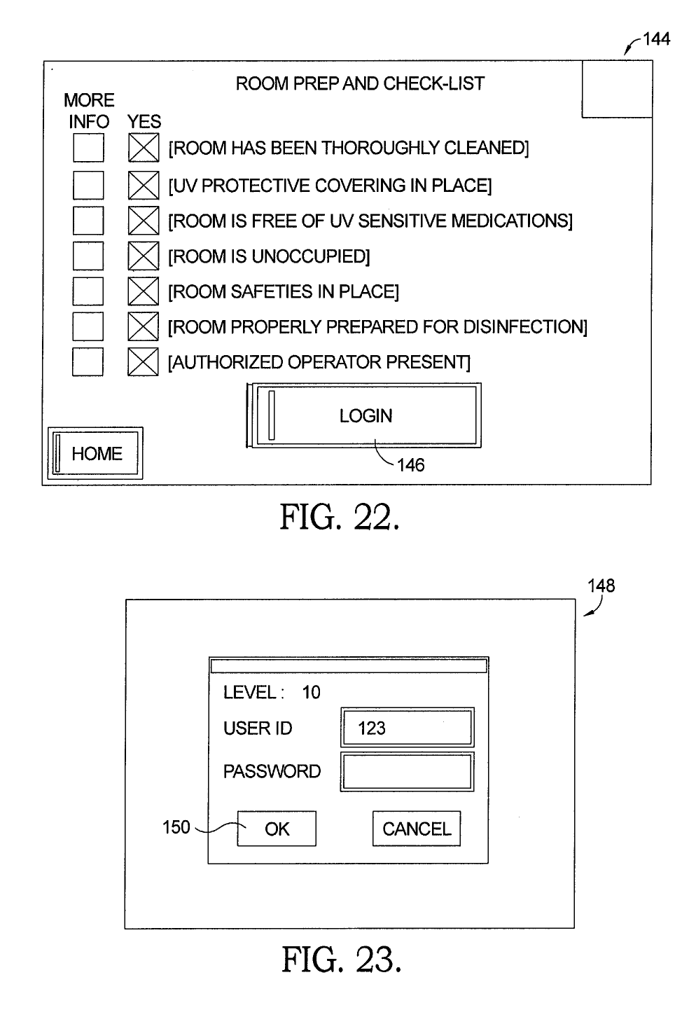

FIGS. 14 through 25 are sequential menu screens of the control station for use by an operator during operation of a UV surface disinfection system in accordance with the present invention; and

FIG. 26 is a block diagram generally illustrating a computing environment in which the various aspects of the present invention may be implemented.

The exemplifications set out herein illustrate various embodiments of the invention, and such exemplifications are not to be construed as limiting the scope of the invention in any manner.

### DETAILED DESCRIPTION OF THE INVENTION

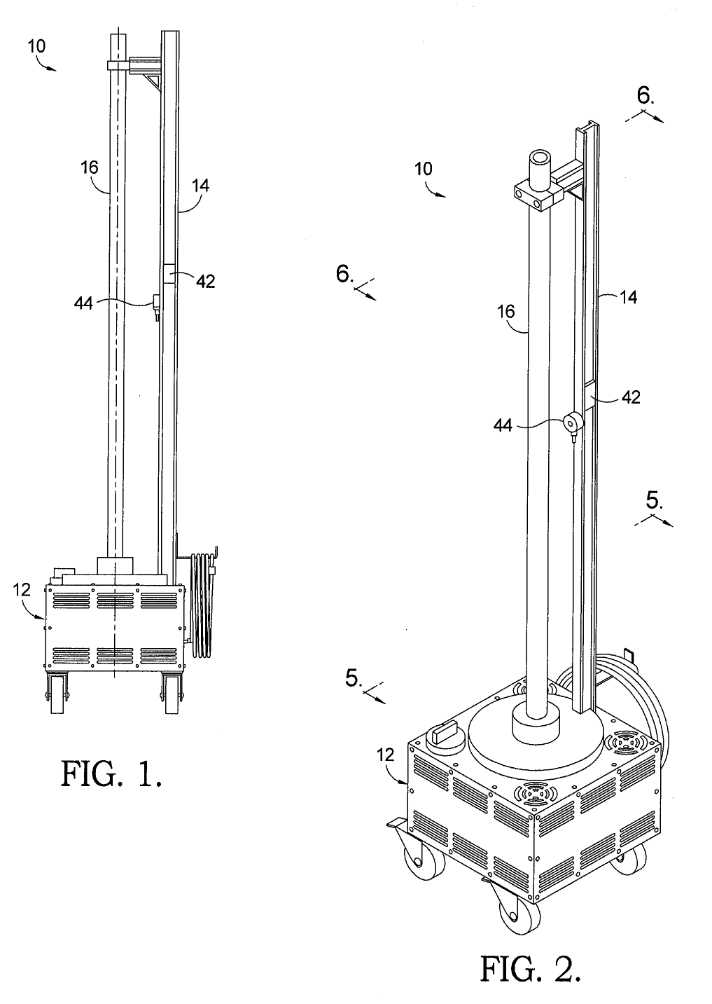

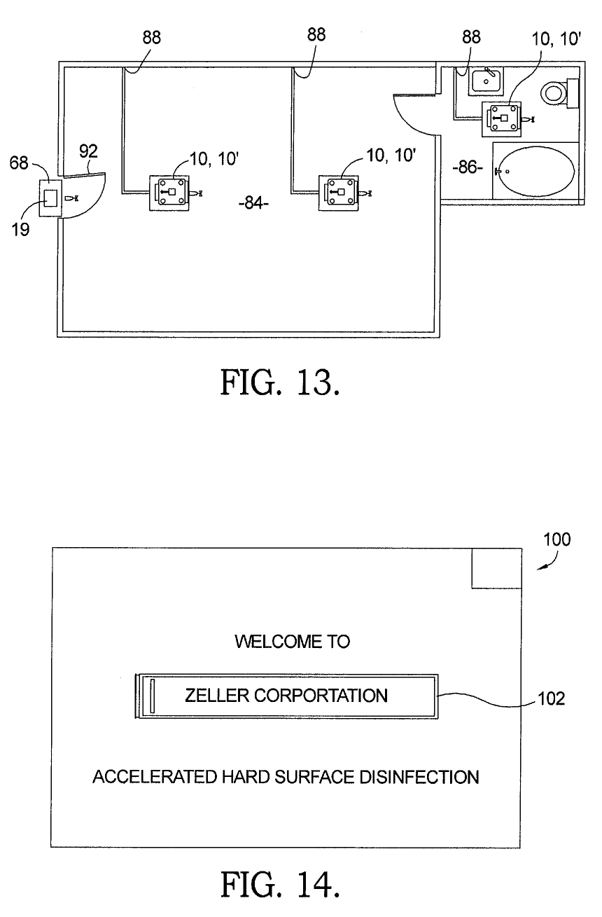

Referring to the drawings in more detail, and initially to FIGS. 1 through 6, one embodiment 10 of an ultraviolet light emitting assembly (ULA) in accordance with the present invention generally comprises a base unit 12, a vertical support rail 14 mounted to base unit 12, a transparent protective sleeve 16 supported by support rail 14, and a UV-C light emitting source 18 positioned within sleeve 16. In accordance with one aspect of the present invention, a plurality of ULAs may be associated with a control station 19 (FIG. 7) to form a system for disinfecting an area, such as a room in a hospital or clinic. In operation of the system, the plurality of ULAs are strategically placed within a room to be disinfected so that, when control station 19 operates to remotely activate and deactivate the plurality of ULAs to emit UV-C light within the room, the shadows within the room are minimized and the surfaces within the room are efficiently disinfected.

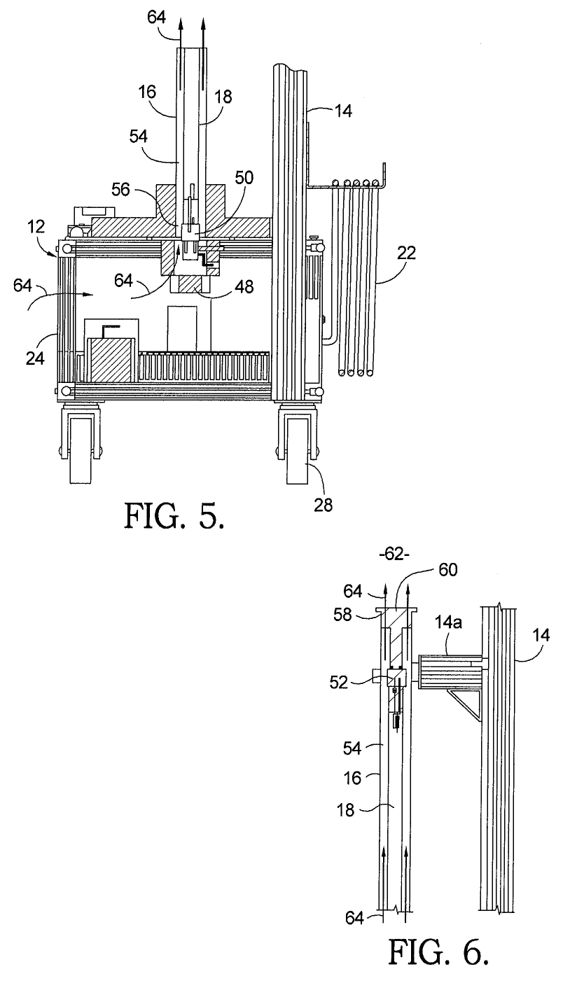

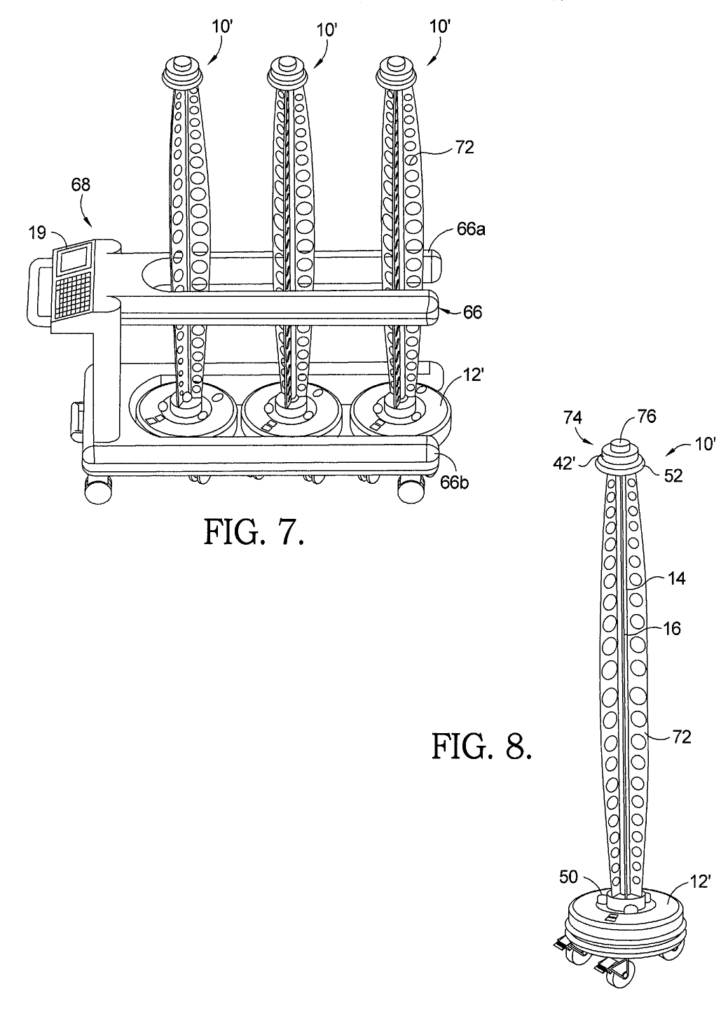

As best seen in FIGS. 3 and 4, base unit 12 may include an enabling on/off safety lock-out switch 20, an electric power cord 22, and a detachable case 24 for enclosing the components of base unit 12. Case 24 may include a plurality of venting slots 26 defined therein for allowing air to flow into and out of the internal compartment of base unit 12. Base unit 12 may be supported on four lockable castors 28 so that the ULA 10 may be easily transported.

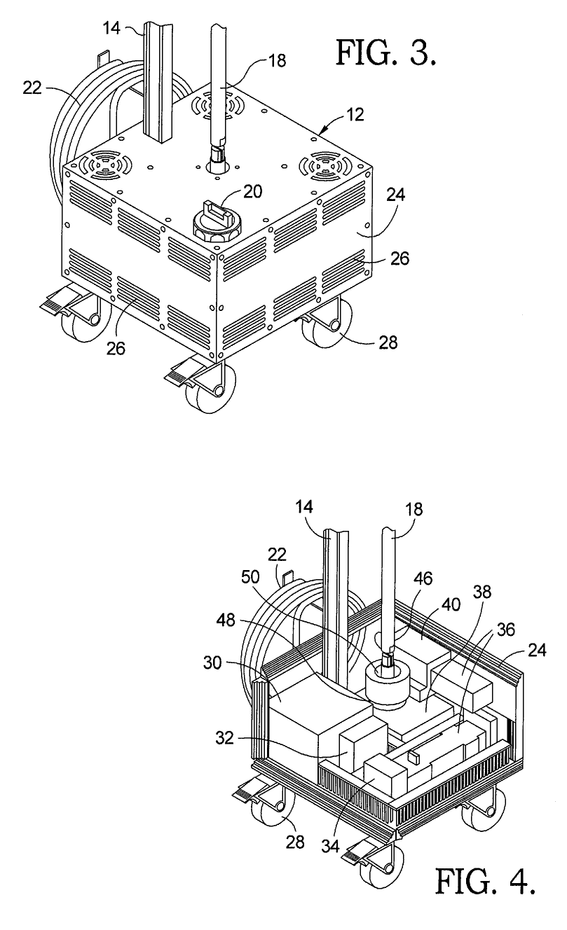

Referring to FIG. 4, base unit 12 may also contain a transformer 30 connected to power cord 22, a DC power supply 32, a contactor 34, a plurality of terminal blocks 36, a ballast 38 and a programmable logic controller (PLC) 40 that monitors and controls the operations of ULA 10 and also transmits status data and other data wirelessly using a Bluetooth antenna or other type of wireless communication device 42 to control station 19 for monitoring by an operator of the system. For example, as best seen in FIG. 2, a UV sensor 44 may be mounted to support rail 14 and configured for measuring fluence levels. UV sensor 44 monitors the output level of the lamp and communicates with PLC 40 to confirm that the system is emitting the required amount of UV-C radiation to disinfect the required surfaces. An audible alarm may be included on base unit 12 and activated by PLC 40 if the UV-C output level falls below a predetermined minimum threshold. Transformer 30 provides the variable-output 220-volt AC power required for lamp ballast 38. Ballast 38 controls the electric power to lamp 18 and is interfaced to PLC 40 preferably via an RS 422 link. PLC 40 uses this link to control ballast parameters. DC power supply 32 provides the 24-volt DC power required for the control of circuits driving lamp 18. Contactor 34 controls the on/off state of the UV-C lamp 18 by switching the power to ballast 38. The coil of contactor 34 is controlled by PLC 40.

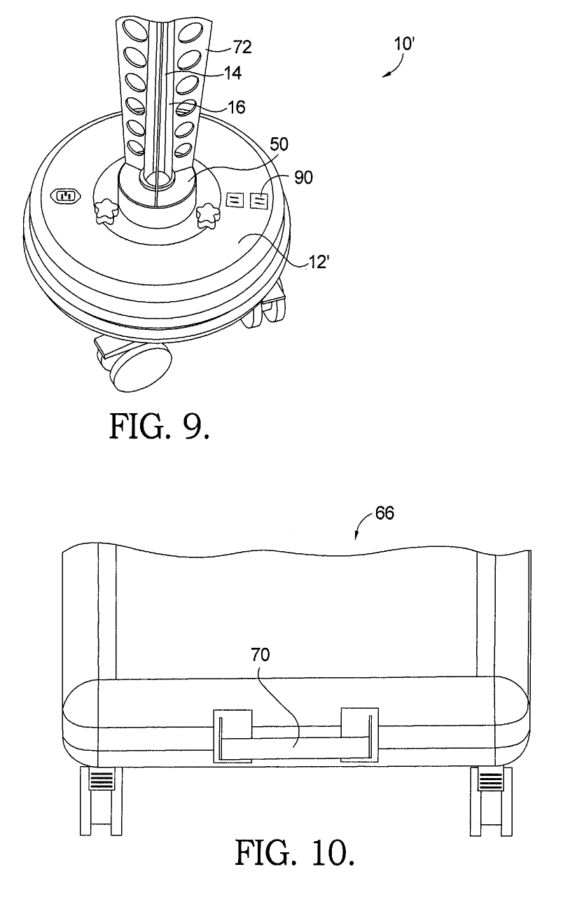

As best seen in FIGS. 4 and 5, base unit 12 may also include a Resistance Temperature Detector (RTD) temperature sensor 46 and a ventilation fan 48. The PLC 40 also monitors and controls the temperature of the glass surface at each lamp's 18 amalgam pellet by selectively enabling ventilation fan 48. The temperature of this location on the UV-C lamp 18 should be monitored in order to maintain stability in the UV-C irradiance level. The RTD/temperature sensor 46 provides glass surface temperature to PLC 40 as part of a feedback control loop including fan 48. For example, if the temperature of lamp 18 rises above a predetermined operable threshold, PLC 40 may be configured to activate fan 48 to lower the temperature of lamp 14 within an acceptable temperature range. Also, PLC 40 may be configured to enable ventilation fan 48 to maintain the temperature of lamp 18 within a predetermined operable temperature range.

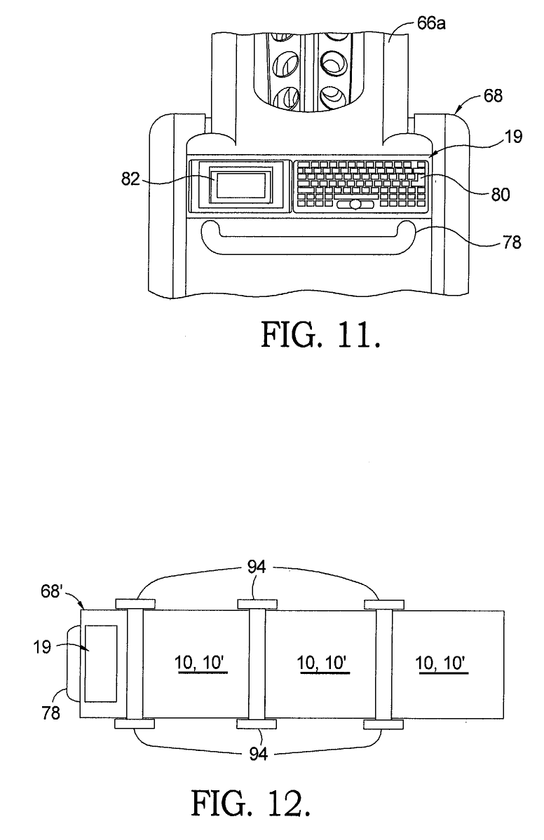

In particular, as best seen in FIGS. 5 and 6, lamp 18 is mounted between a lower socket 50 and an upper socket 52 of ULA 10, and protective sleeve 16, which may be formed of doped quartz glass, is configured so that it surrounds lamp 18 to define a ventilation channel 54 therebetween. A bottom portion of sleeve 16 is configured to be removably secured within an aperture formed within a top portion of base unit 12, and a top portion of sleeve 16 is configured to be removably secured to a top end 14a of support rail 14. Ventilation channel 54 includes a first open end 56 adjacent to lower socket 50 and a second open end 58 adjacent to upper socket 52. Further, a ventilation hub 60 is disposed on the top portion of sleeve 16 to maintain spacing between protective sleeve 16 and lamp 18. Hub 60 has holes defined therein so that second open end 58 remains open to the external environment. Fan 48 is mounted within base unit 12 and positioned so that when fan 48 is activated by PLC 40, fan 48 will pull air through venting slots 26 and blow it through first open end 56, across the surface lamp 18, and out second open end 58 to reduce the temperature of lamp 18, or maintain the temperature of lamp 18 within a predetermined temperature range, as shown by the arrows 64 in FIGS. 5 and 6.

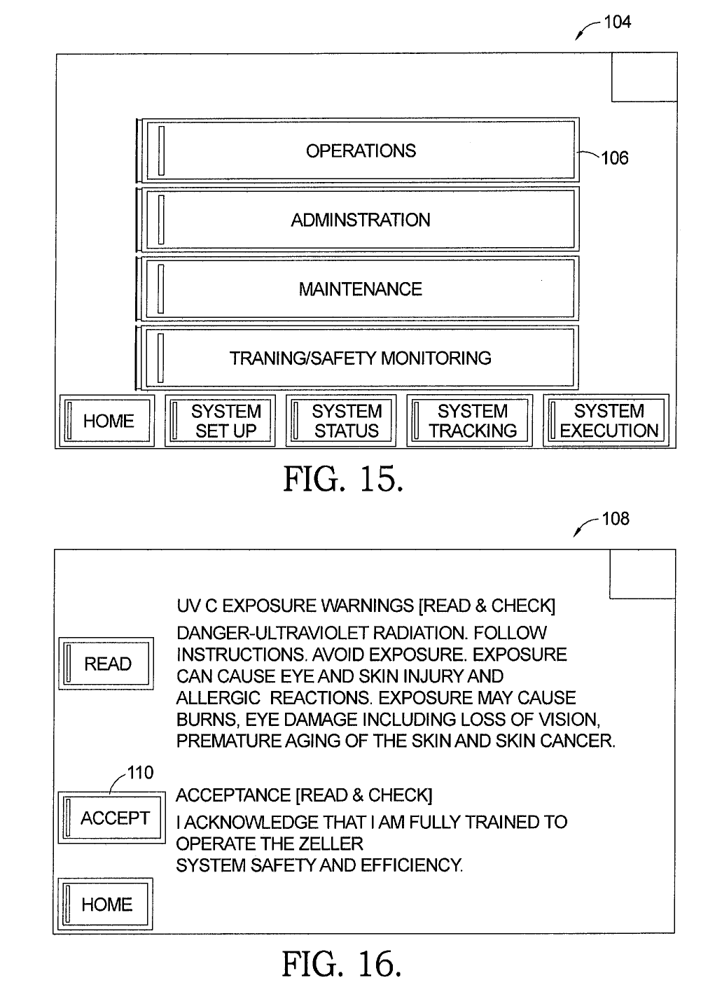

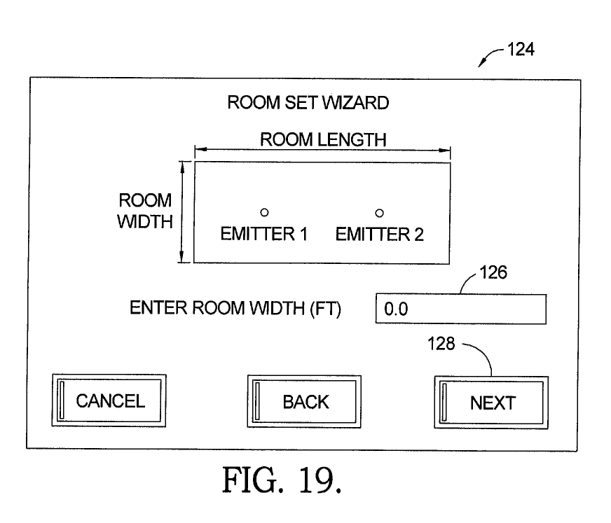

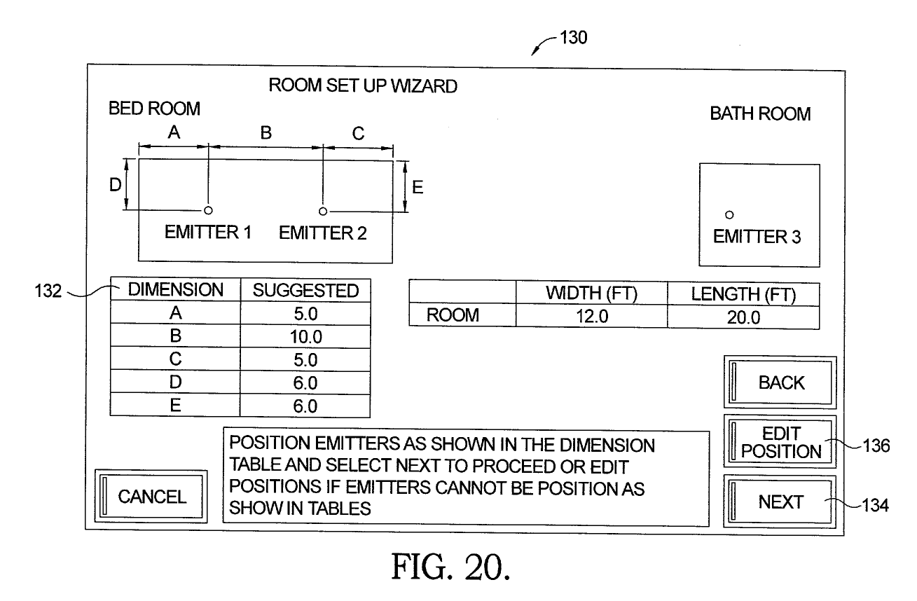

Referring now to FIGS. 7-9, another embodiment 10′ of a ULA in accordance with the present invention is shown. With particular reference to FIG. 7, three ULAs 10′ are shown disposed in a slotted dock 66 having upper and lower portions 66a, 66b formed in a portable transportation and storage docking cart 68. Lower portion 66 may be configured so that ULAs 10′ may be elevated off the ground when positioned within lower portion 66 to facilitate the transportation of the ULAs 10′ from one location to another. As best seen in FIG. 10, ULAs 10′ may be elevated off the ground by actuating a pump action foot pedal 70 mounted to lower portion 66b, which may in turn be released to lower ULAs 10′ back to the ground when seeking to re-deploy ULAs 10′. For convenience in unloading and loading ULAs 10′ from cart 68, the base units 12′ are preferably circular rather than rectangular as in previously described embodiment 10, although the previously described embodiment 10 may also be used with docking cart 68. In embodiment 10′, support rail 14 is omitted and is replaced by a plurality of thin radial flanges 72, which may be fixed to lower socket 50 or base unit 12′, supportive of upper socket 52, and protective of sleeve 16 and UV lamp 14. Moreover, ULA 10′ may include a top assembly 74 including upper socket 50, a motion sensor 76 so that the UV-C light can be shut off if motion is sensed in a room which ULA 10′ is present and actively emitting UV-C light, and a wireless communication device 42′ allowing for communication between ULA 10′ and control station 19. As best seen in FIG. 11, computerized programmable control station 19 may be mounted adjacent to a handle 78 of docking cart 68. Control station 19 includes an operator interface such as a keyboard/touch pad 80 and a screen 82, which may be a touch screen, to assist the operator in controlling the ULAs, and monitoring the ULAs and the conditions within the room, when disinfecting a room using the system. It should be understood that control station 19 may be used with either ULA 10, ULA 10′, or a combination thereof.

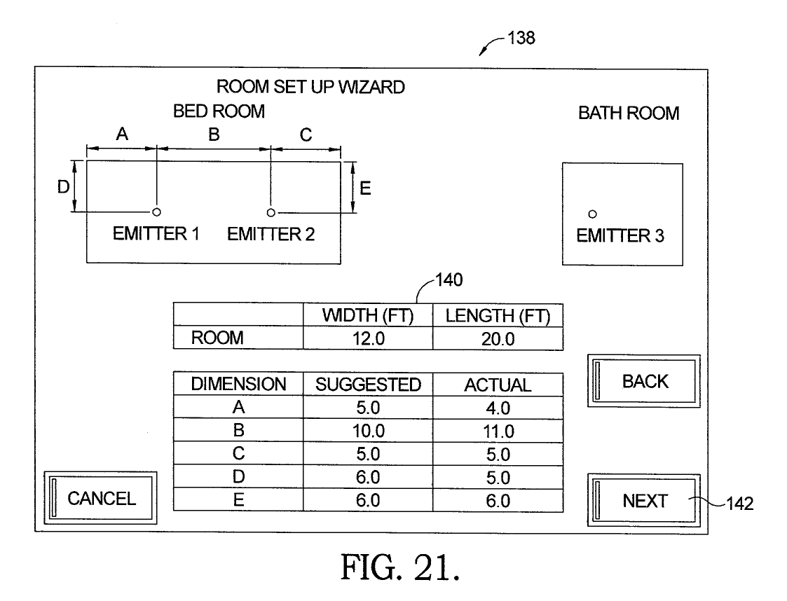

As best seen in FIG. 12, an alternative type of cart 68′ is contemplated wherein a plurality of ULAs 10 or 10′ may be connected to cart 68′ to form a train. The portion of cart 68′ having control station 19 would have handle 78 for pushing and pulling the train of ULAs around the hospital, for example. The cart 68′ and ULAs may be selectively coupled together using a coupling mechanism 94 so that they become a ridged single unit when they are coupled together. The coupled train would move similar to docking cart 68 with multiple wheels as there would be no articulation between ULAs. In operation, coupling mechanisms 94 would be removed to disengage the train so that ULAs may be strategically positioned within the room to be disinfected, and the cart 68′ along with the control station 19 would be positioned in front of door 92 (FIG. 12). One reason for using the train approach instead of the docking approach is that the detached cart 68′ would be significantly smaller than the fully assembled train with its cart 68′ and ULAs or the lift mechanism style of docking cart 68. This is important in a hospital environment since leaving equipment left in the working hall ways is discouraged.

The selection of the UV-C light source to be used in lamp 14 for the ULAs is highly important to the success and acceptance of the system. The following types of UV-C light sources are commercially available: low pressure mercury standard output; low pressure mercury high output; low pressure amalgam; and medium pressure mercury.

Low-pressure UV lamps are sub-divided into three categories: standard output, high output, and amalgam. Standard and high output lamps rely on mercury vapor, which emits UV radiation when excited by electricity. Amalgam lamps are different in that they do not use liquid mercury but rather a mixture of bismuth, indium-2 and mercury (amalgam) that is bound to the inside wall of the lamp's glass envelope (amalgam spot).

Standard and high-output UV lamps' mercury vapor pressures within their glass envelopes are governed by the temperature of tiny droplets of free flowing liquid mercury. The liquid mercury droplets collect at the coldest spot inside the lamp, and maximum UV-C output occurs when the mercury droplets are at a peak temperature of 40° C. The difference between standard and high output lamps is that high output lamps are driven at a higher electrical current and input wattage, are equipped with heavier filaments, and are capable of carrying higher electrical loads. These “heavy duty” filaments also provide a more controlled cold spot (behind themselves) for mercury collection, enabling the lamp to produce greater levels of useable UV-C power (typically two times the power level of a Standard Output UV Lamp).

Amalgam Lamps use a “fixed amalgam spot” in place of free flowing liquid mercury. This “amalgam spot” provides the optimum mercury vapor pressure at an operating temperature of 80° C. which allows for greater input current loads and, in turn, greater UV-C output. Additionally, the “amalgam spot” regulates the mercury during operation: if the internal lamp pressure falls, the “amalgam spot” releases mercury into the excited vapor; if the pressure rises, it will absorb mercury from the vapor. This inherent regulation maintains stable UV output while offering significantly broader ambient temperature tolerances (typically two times the power level of a high output UV lamp).

Medium-pressure lamps produce the majority of their UV output in the UV-A and UV-B spectral areas well outside the specific UV-C wavelength commonly used for disinfecting surfaces. In order to obtain maximum life from a medium-pressure lamp, the lamp's operating temperature must constantly be monitored and controlled. This increased level of control adds a higher level of sophistication and expense to the disinfection system.

ULA 10 or 10′ will be required to operate in existing patient care areas using the existing electrical utilities. This restricts the amount of power the system will have for its operation. Therefore the most important factor in selecting a light source will be the ratio of input power to the corrected output UV-C power of the light source. It is important to select a light source that is the most efficient in generating the raw disinfection energy as this will be a major factor in the time the system takes to disinfect an area.

The system must be designed to disinfect an area repeatedly under operating conditions that will degrade the UV-C output power of the light source over time. The corrected output UV-C power must be used so that the system is designed using the worst case UV-C power output, thereby ensuring that the repeatability of the system is not compromised.

The light source's corrected output UV-C power will be affected by source-dependent variables including ambient temperature and UV losses due to solarization of the lamp tube over operating time, and by source-independent variables including dirt and dust on the lamp surface, transmissivity of air, suspended solids/dust in air, and surface distance from the light source.

The low pressure amalgam light source is the most efficient in generating raw disinfecting UV-C energy. It is 25 percent better that the low pressure standard light source, due mainly to a coating used to reduce the effects of solarization. During lamp operation, the mercury that is used to create the UV energy reacts with the applied electrical arc which, over time, forms a very gradual plating of mercury oxide onto the inside surface of the lamp's glass envelope which absorbs UV light, reducing the transmissivity of the envelope and thereby reducing the disinfection capability of the system over time.

The surface temperature of the various lamp types are different and will also affect the radiation output under different ambient temperatures. The surface temperature of the lamp is also a safety concern as a lamp could become a fire hazard or cause a burn to operating personnel. Preferably, each lamp 18 is sheathed in quartz sleeve 16 that is transparent to UV-C light and provides protection for the lamp 18. Further, sleeve 16 provides ventilation channel 54 between sleeve 16 and lamp 18 for passage of air from cooling fan 48 over the lamp surface to keep lamp 18 cool. Low pressure amalgam lamps have a normal surface temperature operating range of about 80° C. to about 90° C. with a maximum temperature of 130° C. They also have the lowest losses over the largest ambient temperature range, although this should not be a major factor as it is expected that the room temperature will remain substantially constant during disinfection.

The selection of a lamp type will have a maintenance impact on the disinfection system. The quantity of lamps needed for the rated input power will affect the maintenance of the system by affecting cleaning time, lamp replacement number and time, quantity of hazardous material (mercury or amalgam), component failure probability (lamps, ballast, and connections), and durability to breakage. Further, low pressure amalgam lamps require 25 percent fewer components (lamp, ballast, and connector) resulting in lower maintenance. Cleaning of the lamps is the major preventive maintenance issue as it directly impacts the level of UV-C emission from the ULA. The lamps should be kept clean and free of dust at all times, as accumulated dust will absorb UV light and convert it to heat, therefore lowering the effectiveness and lifetime of the UV lamp. For all these reasons, the presently preferred UV-C lamp for use in a disinfection system in accordance with the present invention is a low pressure amalgam lamp.

Referring now to FIG. 12, an exemplary area to be disinfected by a disinfection system in accordance with the present invention may be a room 84, which may be a hospital or clinic room and which may include a dedicated bathroom 86. Embodiments 10 and 10′ define ULAs that can be deployed and controlled around rooms 84, 86 independently of each other.

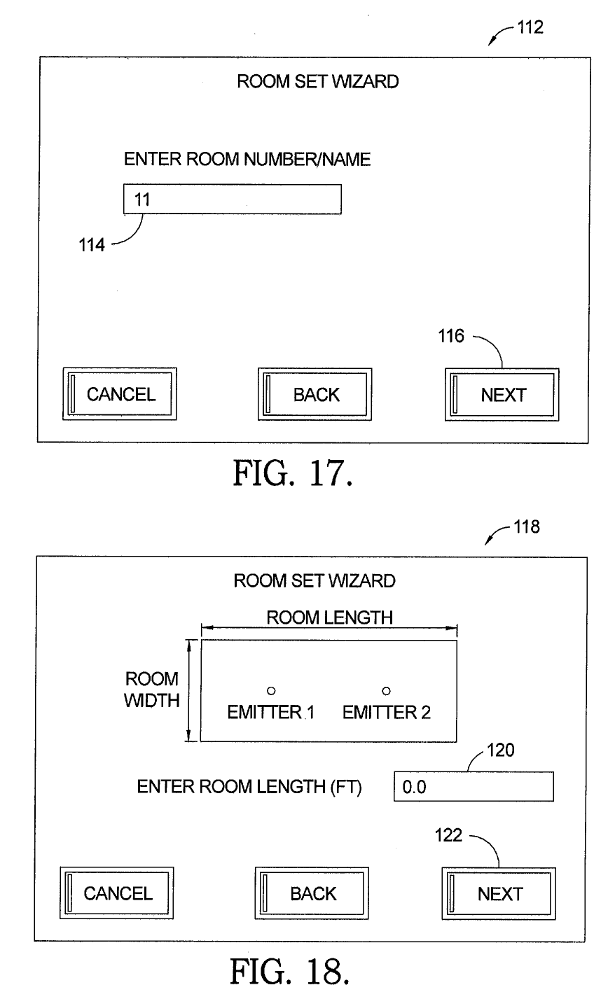

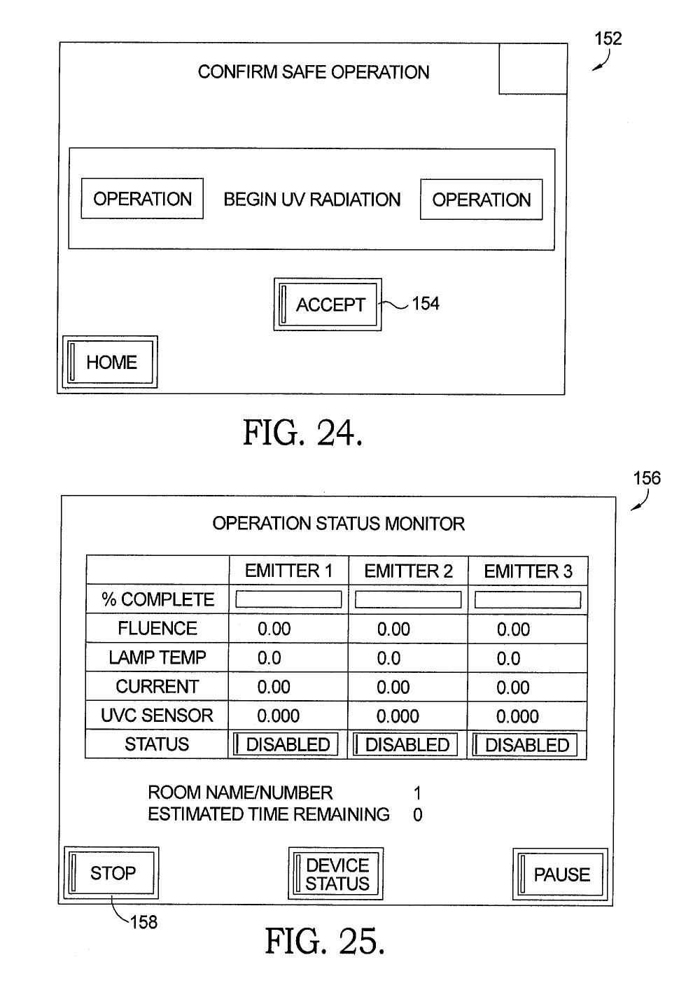

A plurality of ULAs 10 or 10′ are unloaded from docking cart 68, are positioned at strategically determined locations within rooms 84, 86, plugged into room electrical receptacles 88, and enabled by turning on their individual on/off safety lock-out switches 20 (FIG. 3). Alternatively, if room 84 lacks sufficient electrical receptacles, each ULA may be provided with a pass-through circuit and receptacle 90 (FIG. 9) such that a ULA may be plugged into and obtain power from an adjacent ULA. The position of each ULA will be discussed in more detail below. The cart 68 and associated control station 19 then are removed from room 84 and positioned remotely outside the area to be irradiated. Room door 92 is closed and preferably cart 68 is positioned in front of the door 92 such that any physical entry to room 84 during irradiation is temporarily prevented. The further operation of the system is described in detail below in connection with FIGS. 14 through 25.

A currently preferred sequence for safely operating an area disinfection system in accordance with the present invention is as follows. The system is deployed and controlled using an interactive “Deployment Wizard” for an operator via keyboard/touch pad 80 and screen 82 located on control station 19. This wizard leads an operator throughout the steps necessary to position the ULAs 10 or 10′ optimally, and to control and monitor the system's operation.

To initiate the Deployment Wizard, as best seen in FIG. 14, the operator may touch the “Zeller” (or any other system-identifying) Button 102 on the first screen 100. As seen in FIG. 15, a Mode Selection Screen 104 appears, and the operator selects the Operations Button 106 to access the Deployment Wizard. As best seen in FIG. 16, a Deployment Wizard Warning Screen 108 appears where the operator must acknowledge the warning messages by touching the Accept Button 110. As best seen in FIG. 17, a Room Set Wizard Screen 112 appears, wherein the operator must enter the number or name of the room to be disinfected in the Entry Field 114, and then touch the Next Button 116. As best seen in FIG. 18, a Room Set Wizard Screen 118 appears and the operator must enter the length of the room to be disinfected in the Length Entry Field 120, and then touch the Next Button 122. As best seen in FIG. 19, a Room Set Wizard Screen 124 appears and the operator must enter the width of the room to be disinfected in the Width Entry Field 126, and then touch the Next Button 128. As seen in FIG. 20, a Room Set Wizard Screen 130 appears, wherein the operator can touch the Next Button 134 if he has deployed the ULAs 10 or 10′ in the positions as suggested in the Table 132, including the positioning of a ULAs in the Bath Room in a generally central location. The system calculates the suggested placement locations for each ULA based on an algorithm that minimizes the distance from the nearest ULA to the outside walls of the room. The irradiance level at the outside walls of the room is proportional to the inverse square of the distance between the ULA lamp and the surface being disinfected. The system uses the actual distances from the outside walls to calculate the disinfection time, using at least the following factors, in order to obtain the fluence level required to disinfect the room: maximum distance to walls; irradiance level of the lamp at 1 meter distance; and fluence level set point required for disinfection.

If the ULAs 10 or 10′ cannot be deployed as suggested in Table 132, the operator should touch the Edit Positions Button 136 to advance to Room Setup Wizard 138 as seen in FIG. 21, wherein the operator is asked to enter the actual locations of the ULAs 10 or 10′ in Column 140. It should be understood that operation should position ULAs to minimize creation of shadows in the room. The Next Button 142 should then be touched to advance through Wizard.

As best seen in FIG. 22, a Room Prep and Check-list Menu 144 appears and the operator is required to check off that each item on the list has been performed: that the room has been thoroughly cleaned; that UV protective covering where necessary is in place; that the room is free of UV-sensitive medications; that the room is unoccupied; that room safeties, including sealing against entry, are in place; that the room overall is properly prepared for disinfection; and that an authorized operator is present. The operator then presses the Log In Button 146.

As best seen in FIG. 23, a Log-In Menu 148 appears and, before starting a disinfection cycle, the operator is required to enter a user ID and password, then press the OK Button 150. As best seen in FIG. 24, a Confirm Safe Operation Menu 152 appears and the operator is required to press an Accept Button 154 to start a disinfection cycle. As best seen in FIG. 25, an Operation Status Monitor Menu 156 appears. This screen displays dynamic data relative to the operation of the three ULA 10 or 10′ emitters during the disinfection cycle. Should the system go out of control during operation, the operator presses the Stop Button 158 to de-energize all ULAs. In normal operation, the control station 19 will automatically shut down the system at the end of the prescribed disinfection cycle.

As would be appreciated by one skilled in the art, the described Deployment Wizard may be a software module that is individually or collectively implemented on single or multiple computing devices, such as control stations 19, and such computing devices may be co-located or otherwise dispersed across a network 160. Such implementations and arrangements achieve the objectives of the present invention and are contemplated and within the scope of the present invention.

Having described the apparatus and method of the present invention and an embodiment thereof, an exemplary computer environment for implementing the described Deployment Wizard, or the functionality of PLC 40 in each ULA 10 or 10′ as previously described, is set forth below.

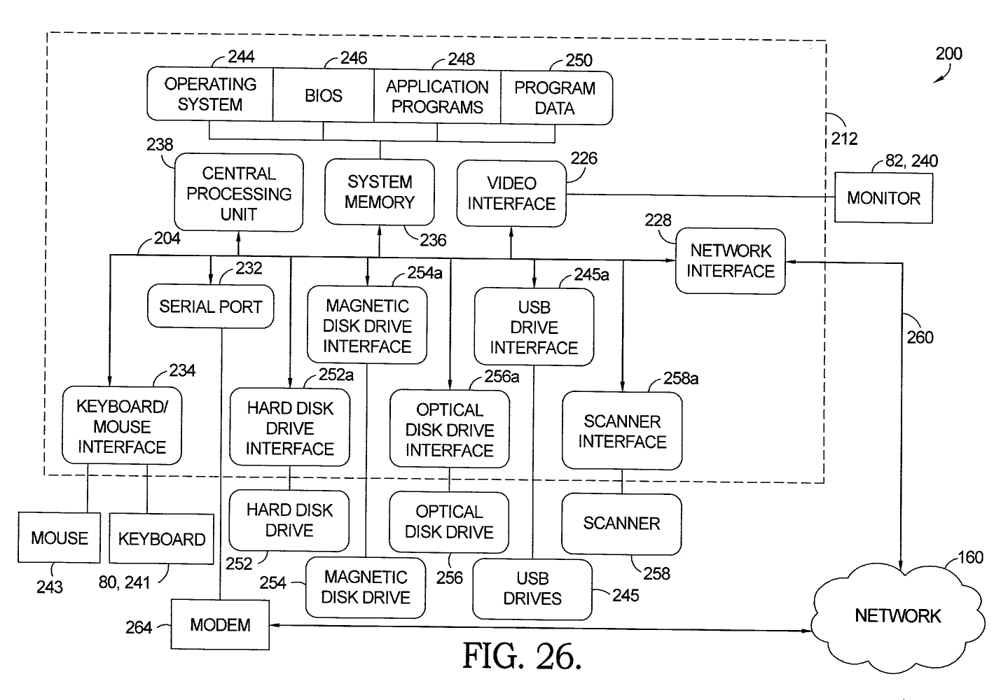

FIG. 26 illustrates an exemplary computing environment 200 that can be used to implement any of the processing thus far described. As shown, computer 212 may be a personal computer including a system bus 224 that couples a video interface 226, network interface 228, one or more serial ports 232, a keyboard/mouse interface 234, and a system memory 236 to a Central Processing Unit (CPU) 238. A monitor or display 82, 240 is connected to bus 224 by video interface 226 and provides the user with a graphical user interface to interact with the system and method of the present invention. The graphical user interface allows the user to enter commands and information into computer 212 using a keyboard 80, 241 and a user interface selection device 243, such as a mouse or other pointing device. Keyboard 80, 241 and user interface selection device are connected to bus 224 through keyboard/mouse interface 234. The display 82, 240 and user interface selection device 243 are used in combination to form the graphical user interface which allows the user to implement at least a portion of the present invention. Other peripheral devices may be connected to remote computer through serial port 232 or universal serial bus (USB) drives 245 to transfer information to and from remote computer 212. For example, personal digital assistants (PDA) or other smart devices may be connected to computer 212 through serial port 232 or USB port 245a so that transaction information or other data may be downloaded to system memory 236 or another memory storage device associated with computer 212.

The system memory 236 is also connected to bus 224 and may include read only memory (ROM), random access memory (RAM), an operating system 244, a basic input/output system (BIOS) 246, application programs 248 and program data 250. Computer 212 may further include a hard disk drive 252 for reading from and writing to a hard disk, a magnetic disk drive 254 for reading from and writing to a removable magnetic disk (e.g., floppy disk), and an optical disk drive 256 for reading from and writing to a removable optical disk (e.g., CD ROM or other optical media). Computer 212 may also include USB drives 245 and other types of drives for reading from and writing to flash memory devices (e.g., compact flash, memory stick/PRO and DUO, SD card, multimedia card, smart media xD card), and a scanner 258 for scanning items to be downloaded to computer 212. A hard disk interface 252a, magnetic disk drive interface 254a, an optical drive interface 256a, a USB interface 245a, and a scanner interface 258a operate to connect bus 224 to hard disk drive 252, magnetic disk drive 254, optical disk drive 256, USB drive 245 and a scanner 258, respectively. Each of these drive components and their associated computer-readable media may provide computer 212 with non-volatile storage of computer-readable instruction, program modules, data structures, application programs, an operating system, and other data for the computer 212. In addition, it will be understood that computer 212 may also utilize other types of computer-readable media in addition to those types set forth herein, such as digital video disks, random access memory, read only memory, other types of flash memory cards, magnetic cassettes, and the like.

Computer 212 may operate in a networked environment. Network interface 228 provides a communication path 260 between bus 224 and the network 160. It will be appreciated by one skilled in the art that the network connections shown herein are merely exemplary, and it is contemplated and within the scope of the present invention to use other types of network connections between computer 212 and any other computing devices including both wired and wireless connections.

From the foregoing, it will be seen that this invention is one well adapted to attain all the ends and objectives hereinabove set forth together with other advantages which are obvious and which are inherent to the method and apparatus. It will be understood that certain features and sub-combinations are of utility and may be employed without reference to other features and sub-combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. As used herein, the terms “having” and/or “including” and other terms of inclusion are terms indicative of inclusion rather than requirement.

While the invention has been described with reference to preferred embodiments, it will be understood by those skilled in the art that various changes may be made and equivalents may be substituted for elements thereof to adapt to specific situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

What is claimed is:

1. An ultraviolet light emitting assembly for disinfecting surfaces within an area, the assembly comprising:
a base unit including a programmable logic controller and a ventilation fan;
a support rail mounted to said base unit;
a transparent sleeve supported by said support rail;
a UV-C light emitting source positioned within said sleeve defining a ventilation channel between said sleeve and said UV-C light emitting source, said ventilation channel including a first opened end and a second opened end; and
a temperature sensor associated with said UV-C light emitting source for monitoring the temperature of said UV-C light emitting source,
wherein said ventilation fan is positioned within said base unit for blowing air into said first opened end of said ventilation channel,
wherein said programmable logic controller is communication with said temperature sensor, said programmable logic controller being configured for enabling said ventilation fan to maintain the temperature of said UV-C light emitting source within a predetermined temperature range, or enabling said ventilation fan if the temperature of said UV-C light emitting source exceeds a predetermined threshold, and
wherein said programmable logic controller is configured for selectively enabling said UV-C light emitting source to radiate UV-C light into the area to disinfect the surfaces located within the area

2. The assembly in accordance with claim 1 wherein said UV-C light emitting source is an amalgam lamp.

3. The assembly in accordance with claim 1 wherein said sleeve is formed of quartz.

4. The assembly in accordance with claim 1 further comprising a UV sensor mounted to said support rail and in communication with said programmable logic controller.

5. The assembly in accordance with claim 1 further comprising an antenna for allowing said programmable logic controller to wirelessly communicate with a remotely positioned control station.

6. The assembly in accordance with claim 1 wherein said temperature sensor is a Resistance Temperature Detector (RTD).

7. The assembly in accordance with claim 1 further comprising a motion detector that is in communication with said programmable logic controller.
